SEQUENCE LISTING (8 sequences)

SEQ ID NO: 1 - DNA, 1569 bp, Homo sapiens
gggtcgggcagcgccgccctccctctccccctgtcctcggaggggtcgaaggcgccggggccccggggcgctgggggctgcagtgcgggcctggggagggcgcctgcgcgtcgggcagcccaggctcggtattctgtcgaatggaggaacctcaccttggatgtccccatggagccttggagggatgcagaagacaggattggccagcgacagtcaaagtggccggcacccccctctaccagctccccagttcctggaggctgggggccaaaagaaagacacaggctactttgacaacctgaaagcgaactccagaaatatcaccaacagcatgacctttttgaccaaatccagcaaccagagctttatggttttccacaataaggttcaagcaaccatcactgaatacaaaggctgtgattttcttgccatccttgatccactggaccctgacacacttcctaatggcagaatttggctttttggcttcaactcctacttttcccagcacaattccttttgcatgagaagcacctccaaaagggttggccttcagggctgtgcccaaatgggctttcttgtactgtttatcatgccacttctgatcttgttggtgactacagagactcctagcagtatgaggtccacgacacttgcccatcctgcagtgctacgggcctgagcaaaagagagaagcagctgtcccagcctggcgtggcggcacaagccagcagtcccagctactcaggaggctgaggcaggagaatcacttgagcctggaaggcagaggttgcaatgagtcaagatcgtgtcattgcactccaacctgggtgaccgagtgagactccatctcaaaataaagaaaaaaaagaaaagaaattcagcaaatgaaatgcaaactctatactctaaaagctgcaaacattattgaaagaaattacagaagatctaaacaaatggaaagacattccatattcatggattggaagacttaataaaatggcagtatccccaaattgatctacagattcaacataattcctactgaaatcacagaggcttctttgcagaaactgacaggttgatgctaatagccaaaataatcttgaaagaaaaagaacaaagtctgaaggccgggtgcagtgactcatacctgtaatctcagtgctttggaaggttgaggtggaaggattgcttgaggccaggagttagagaccagcctgggcagtataacaagactcctgtctctataaaacattaaaaaaaaaattaaccaggcatggtggcgcatgtctgtaatcccagttactcagagaggctgaggtgggaggatcacttgagccaaggagtttgaggctgccgtgagccgtgatcgtgccactctcccctgggtgacagtgtgagacagtatctgaagaaaaaaaaaattcatttccactgcagaaattgcctgcagtgggaatttctgtgcttgagtctatggtacctgccacaggggaatttacattgcttttactattggaattttgtgatcttcttataaaggattaaagacaaacaaggtttatc

SEQ ID NO: 2 - protein, 173 aa, Homo sapiens
MSPWSLGGMQKTGLASDSQSGRHPPLPAPQFLEAGGQKKDTGYFDNLKANSRNITNSMTFLTKSSNQSFMVFHNKVQATITEYKGCDFLAILDPLDPDTLPNGRIWLFGFNSYFSQHNSFCMRSTSKRVGLQGCAQMGFLVLFIMPLLILLVTTETPSSMRSTTLAHPAVLRA

SEQ ID NO: 3 - protein, 22 aa, Homo sapiens
GCAQMGFLVLFIMPLLILLVTT

SEQ ID NO: 4 - DNA, 522 bp, Homo sapiens
atgtccccatggagccttggagggatgcagaagacaggattggccagcgacagtcaaagtggccggcacccccctctaccagctccccagttcctggaggctgggggccaaaagaaagacacaggctactttgacaacctgaaagcgaactccagaaatatcaccaacagcatgacctttttgaccaaatccagcaaccagagctttatggttttccacaataaggttcaagcaaccatcactgaatacaaaggctgtgattttcttgccatccttgatccactggaccctgacacacttcctaatggcagaatttggctttttggcttcaactcctacttttcccagcacaattccttttgcatgagaagcacctccaaaagggttggccttcagggctgtgcccaaatgggctttcttgtactgtttatcatgccacttctgatcttgttggtgactacagagactcctagcagtatgaggtccacgacacttgcccatcctgcagtgctacgggcctga

SEQ ID NO: 5 - DNA, 522 bp, Homo sapiens
atgtccccatggagccttggagggatgcagaagacaggattggccagcgacagtcaaagtggccggcacccccctctaccagctccccagttcctggaggctgggggccaaaagaaagacacaggctactttgacaacctgaaagcgaactccagaaatatcaccaacagcatgacctttttgaccaaatccagcaaccagagctttatggttttcctcaatatggttcaagcaaccatcactgagtacaaaggctgtgattttcttgccatccttgatccactggaccctgacacacttcctaatggcagaatttggctttttggcttcaactcctacttttcccagcacaattccttttgcatgagaagcacctccaaaagggttggccttcagggctgtgcccaaatgggctttcttgtactgtttatcatgccacttctgatcttgttggtgactacagagactcctagcagtatgaggtccacgacacttgcccatcctgcagtgctacgggcctga

SEQ ID NO: 6 - protein, 173 aa, Homo sapiens; identical to SEQ ID NO: 2

SEQ ID NO: 7 - protein, 141 aa, Mouse
MQSQQRNIGYFNHLKADSRNITYSMTFSTKSSNQNFIIFLNEVQAAIIGHERCDLLPVLNELHPDALPDGRIWLFGLNPYFFQHNSLCMRGTPKRIGLQGCAQVGFLVLFVMPLLIPSVTAELPGSSETTTLAHLAGATGP

SEQ ID NO: 8 - protein, 173 aa, Homo sapiens; identical to SEQ ID NO: 2

(19) United States
(12) Patent Application Publication
(10) Pub. No.: US 2010/0286252 A1
(43) Pub. Date: Nov. 11, 2010

(54) HUMAN FG01 GENE AND ITS APPLICATIONS

(75) Inventors: Wu-Bo LI, North Potomac, MD (US); Michael Kinch, Laytonsville, MD (US); Michael Goldblatt, McLean, VA (US)

Correspondence Address: Steven B. Kelber;Berenato & White, LLC, 6550 Rock Spring Drive, Suite 240, Bethesda, MD, 20817, US

(73) Assignee: Functional Genetics, Inc., Gaithersburg, MD (US)

(21) Appl. No.: 12/776,532

(22) Filed: May 10, 2010

Related U.S. Application Data
(63) Continuation-in-part of application No. 12/652,877, filed on Jan. 6, 2010.
(60) Provisional application No. 61/176,530, filed on May 8, 2009.
(60) Provisional application No. 61/179,409, filed on May 19, 2009.
(60) Provisional application No. 61/179,409, filed on May 19, 2009.
(60) Provisional application No. 61/176,530, filed on May 8, 2009.

(51) Int. Cl. A61K 31/7088; C07H 21/04; C07K 14/00; C40B 30/00; C12N 5/02; A61K 48/00

(52) U.S. Cl. 514/44 R; 536/23.1; 530/350; 506/7; 435/375

(57) ABSTRACT

A human gene, fg01, on chromosome 8, is identified, as well as a truncated form on chromosome 5. Upregulation appears to suppress the Alzheimer's phenotype, (AB plaques and hyperphosphorylated tau tangles) which may address the onset of symptoms or progression of symptoms associated with AD. Screening methods are also set forth.

### PRIORITY DATA AND INCORPORATION BY REFERENCE

This application claims benefit of priority to U.S. Provisional Patent Application No. 61/176,530 filed May 8, 2009 and U.S. Provisional Patent Application No. 61/179,409, filed May 19, 2009, which are incorporated by reference in their entirety. This application further claims benefit of priority to U.S. patent application Ser. No. 12/652,877 which is incorporated herein by reference in its entirety.

### BACKGROUND OF THE INVENTION

1. Field of the Invention

This invention involves the identification of a human gene implicated in the development of Alzheimer's Disease (AD) and methods of enhancing expression of that gene or the protein it encodes to inhibit, treat, reduce and possibly reverse symptoms associated with AD. The gene, and the protein it encodes, is found in two forms—a truncated form on chromosome 5, that lacks a transmembrane domain, and a longer or full length form, found on chromosome 8, that includes a transmembrane domain. Both forms may be of both diagnostic and therapeutic value.

2. Related Art

This application is related to the discovery set forth in U.S. patent application Ser. No. 12/399,850 of a mouse gene fg01 that expresses a mouse protein fg01 which appears to inhibit activity of the serine/threonine kinase GSK3. Inhibition or down-regulation of this enzyme has been shown to reduce certain symptoms associated with AD, including the accumulation of Aβ, a cleavage product of APP which is found in plaques and other physiognomy associated with AD. Named inventors herein are also inventors named in 12/399,850 and the disclosure of U.S. patent application Ser. No. 12/399,850 is incorporated by reference herein in its entirety. That application identifies a mouse gene, and the protein it encodes, that were identified by a process called Random Homologous Gene Perturbation (RHGP), which permits random insertion of a gene search vector which, when inserted in the allele of a eukaryotic gene, generates an antisense or sense RNA sequence, which inactivates or activates the matching allele. This process allows inspection of the entire eukaryotic genome of a cell, to identify specific targets for manipulation. It is disclosed in U.S. patent application Ser. No. 11/928,393. Although not essential to the practice of the invention disclosed and claimed herein, the disclosure of this application is also incorporated by reference. In this application, the human gene, a prerequisite for fashioning therapeutic treatment based on that gene and its encoded protein, are set forth. Human fg01 and the encoded human fg01 protein offer opportunities to screen for those patients who can be effectively treated to control the development of AD symptoms, as well as opportunities for therapeutic intervention.

### BACKGROUND OF THE TECHNOLOGY

AD is a progressive and generally fatal neural disease. Symptoms reflected by AD patients include profound memory loss, a reduction in higher order thinking and aberrant behavior. Currently, there is no known cure, although a number of treatments for the symptoms described are being explored. In part due to its progressive nature, the toll taken by AD patients is enormous, on their resources, and those of care-givers.

There are two profound brain structures that are associated with the “AD phenotype.” These are generally referred to as “plaques” and “tangles.” Plaques reflect the excessive production and accumulation of the β-amyloid peptide (Aβ that is the product of cleavage of the protein APP. Genetic and chemical studies have shown that a variety of pathogenic mutations in the APP gene and in genes encoding proteins known as presenilins 1 and 2 (PS1 and PS2), the major component of the gamma-Secretase complex, increase the production of Aβ peptide. A mouse model of AD, where the mice exhibit early onset of plaque formation, and hyperphosphorylation of another protein, tau, which is typically found within dead or dying neuronal cells in the form of tangles, which are comprised largely of tau proteins so phosphorylated that they have been rendered insoluble, and appear in the form of filaments, is disclosed in U.S. Pat. No. 5,898,094. Related research gave rise to a mouse model with a human tau transgene, to better model AD, as set forth in U.S. Pat. No. 7,161,060.

Subsequently, in U.S. patent application Ser. No. 12/399,850, the identification of a gene in mice having a direct correlation with the “AD phenotype” is reported, by the current inventors and others. Although upregulation of the gene and protein encoded thereby, mouse fg01 and mouse fg01, appeared to offer some inhibition of the cleavage patterns that give rise to the AD phenotype, suppressing plaque and tangle formation, extensive research indicated that no human analogue existed. (Neuron, Zhang et al, in press). While transgenic modification of human cells to express the mouse fg01 gene indicated the value of upregulation, the protein encoded by this mouse gene is immunogenic, and does not offer a method for treatment of humans.

### SUMMARY OF THE INVENTION

A sequence for the non-truncated form of human fg01 is set forth in FIG. 1, encoding a 173 amino acid protein, human fg01, set forth at FIG. 2. The protein is a type 1b transmembrane protein, similar in general structure to the mouse fg01. It shares less than 70% homology with the mouse analogue, however, which is the basis for the immunogenicity of the mouse fg01 protein.

A sequence for the truncated from of human fg01 found on chromosome 5 is set forth in FIG. 8, encoding a putative 96 amino acid protein lacking a transmembrane domain, the active form of which may be a methionine initiated 74 residue protein as shown in FIG. 9 (the first methionine is shown in red). Herein, the non-truncated from is referred to as human fg01 and human fg01 (the protein) while the truncated form is indicated as truncated human fg01 and truncated human fg01

Mouse FG01 was identified in an RHGP-based campaign to identify novel regulators of Aβ production. The regulation of Aβ is understood to be a key marker of Alzheimer's Disease (AD) damage and inhibitors of Aβ could provide much-needed opportunities to prevent or treat this disease.

The RHGP campaign utilized a murine cell line and hypothesized a mechanism in which fg01, a transmembrane protein, induces the enzymatic activity of adenylyl cyclase, which promotes the production of cAMP and in turn activates protein kinase A (PKA). PKA activation then serves to inhibit GSK3 kinase activity and thereby prevents the phosphorylation of tau. These activities serve to decrease Aβ production and thus decrease or prevent the deposition of amyloid plaques, the hallmark of the manifestation of Alzheimer's disease. Altogether, these results suggest that upregulation of human fg01 could be used to decrease the cellular pathogenicity of Alzheimer's disease.

Based on these findings, therapeutics that directly upregulate human fg01 expression (e.g., via gene therapy), indirectly upregulate fg01 expression (e.g., inducers of endogenous human fg01 expression or that mimic fg01 expression (e.g., that activate adenylyl cyclase, increase cAMP, inhibit GSK3 or prevent phosphorylation of tau) could similarly have utility for the management of Alzheimer's Disease. By the same token, in alternative scenarios, truncated human may be effective in both a diagnostic role and a therapeutic role in the treatment of AD.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain the features of the invention.

FIG. 1: Human fg01 cDNA sequence. Provides the sequence of human fg01 cDNA obtained from a fetal brain tissue library. The start ATG and stop codon TGA are in red. The intron and exon boundaries are shown in green.

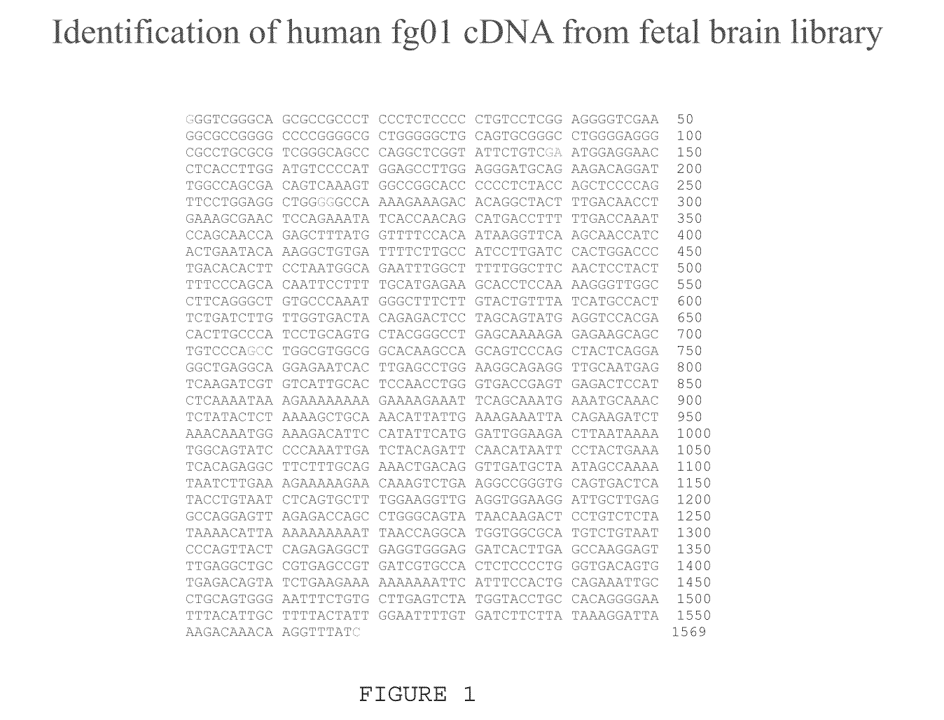

FIG. 2: Human fg01 protein sequence. The human fg01 protein sequence encoded by the fg01 gene is given in FIG. 2, from N- to C-terminus The transmembrane domain of human fg01 protein is given in blue, and several potential glycosylation sites are set forth in red.

FIG. 3: Human fg01 polymorphism. Certain sites in the human fg01 gene are identified as polymorphic, with identified mutations indicated.

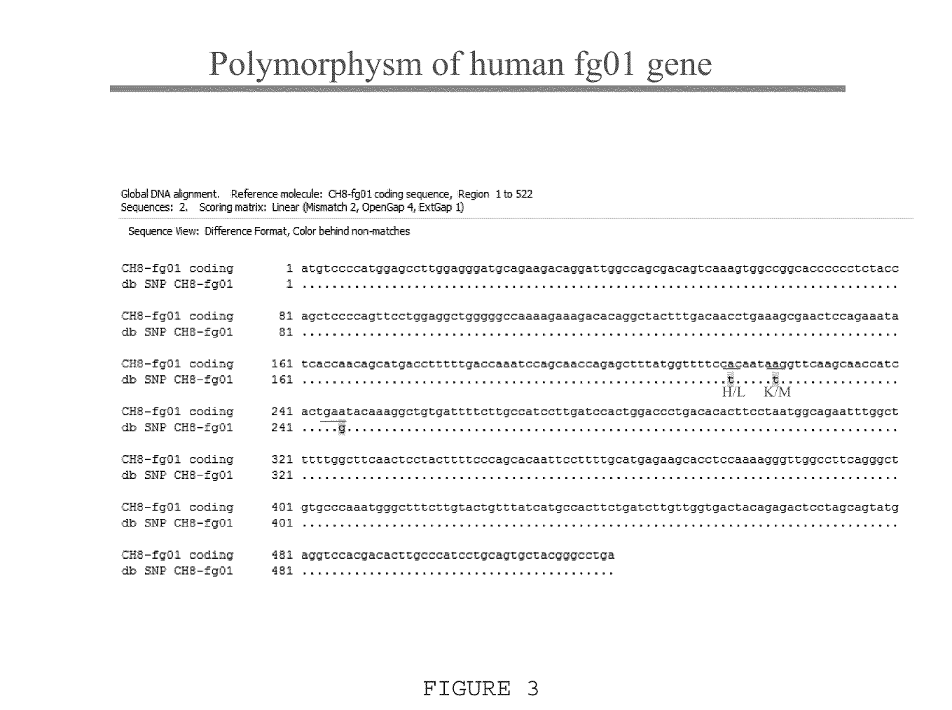

FIG. 4: Potential Phosphorylation Sites. The human fg01 protein is likely activated by phosphorylation. It exhibits a number of phosphorylation sites both intracellularly and beyond the transmembrane domain, in the extracellular portion of the molecule. These potential phosphorylation sites are identified in FIG. 4.

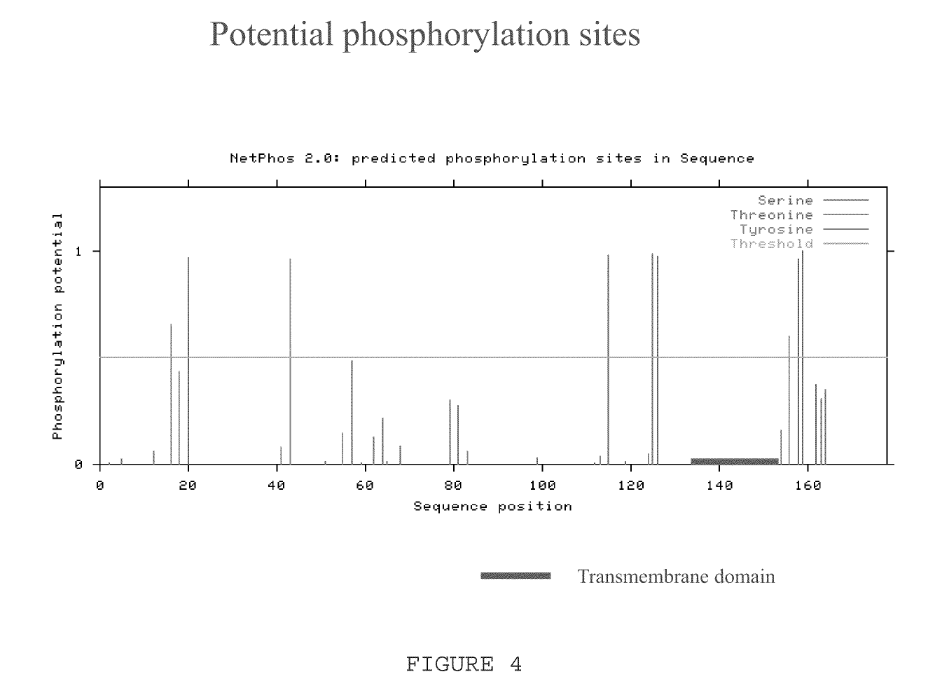

FIG. 5: Alignment with murine fg01. The search for the human fg01 gene was inspired by identification of the murine fg01 gene, described in U.S. patent application Ser. No. 12/399,850 and in Neuron, Zhang et al, (in press). Although those researchers found no human counterpart, an alignment of the human and murine fg01 proteins is set forth in FIG. 5.

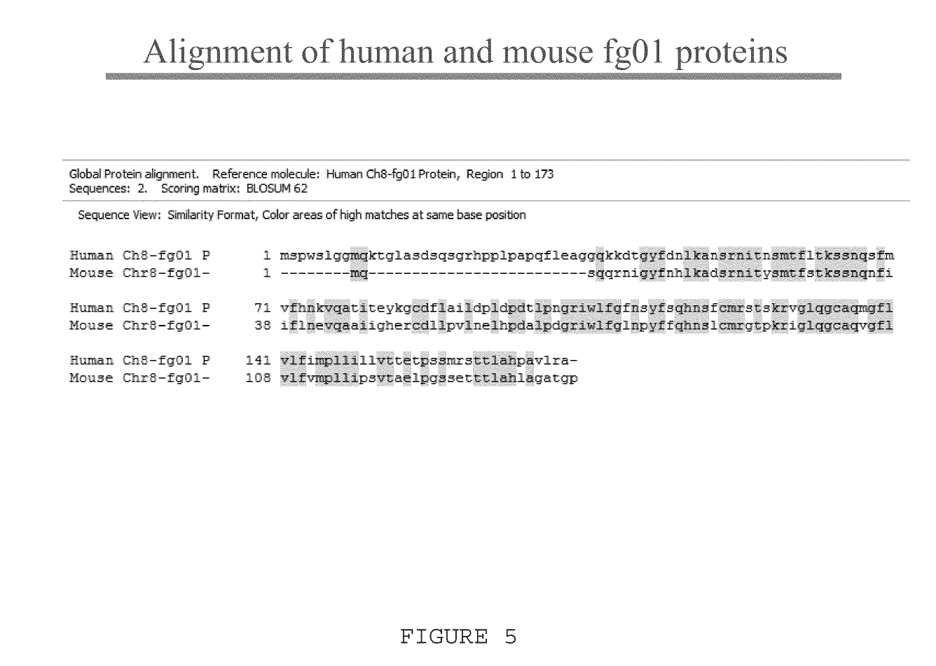

FIG. 6: Structure prediction for human fg01. Based on the sequence of human protein fg01, and the conformance of similar proteins, the conformational structure of human fg01, with amino acid residue identification inserted, is set forth in FIG. 6.

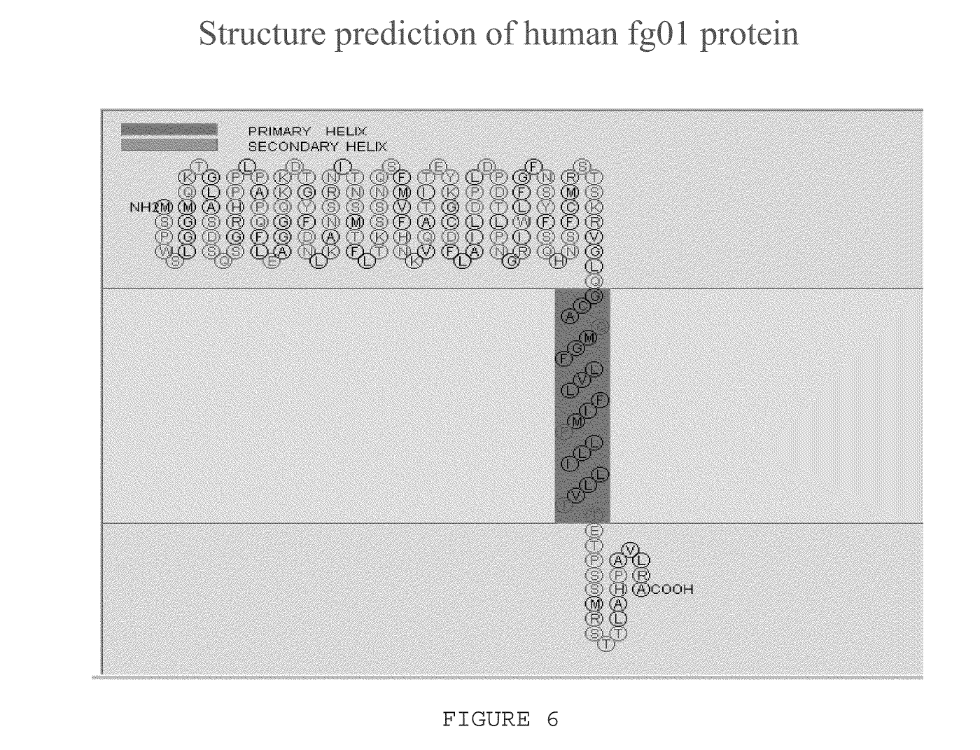

FIG. 7: Structure comparison of human and murine fg01 proteins. The nominal three dimensional structure of the human and mouse fg01 proteins are compared in FIG. 7, showing a conserved structure although the sequence homology is less than 70%.

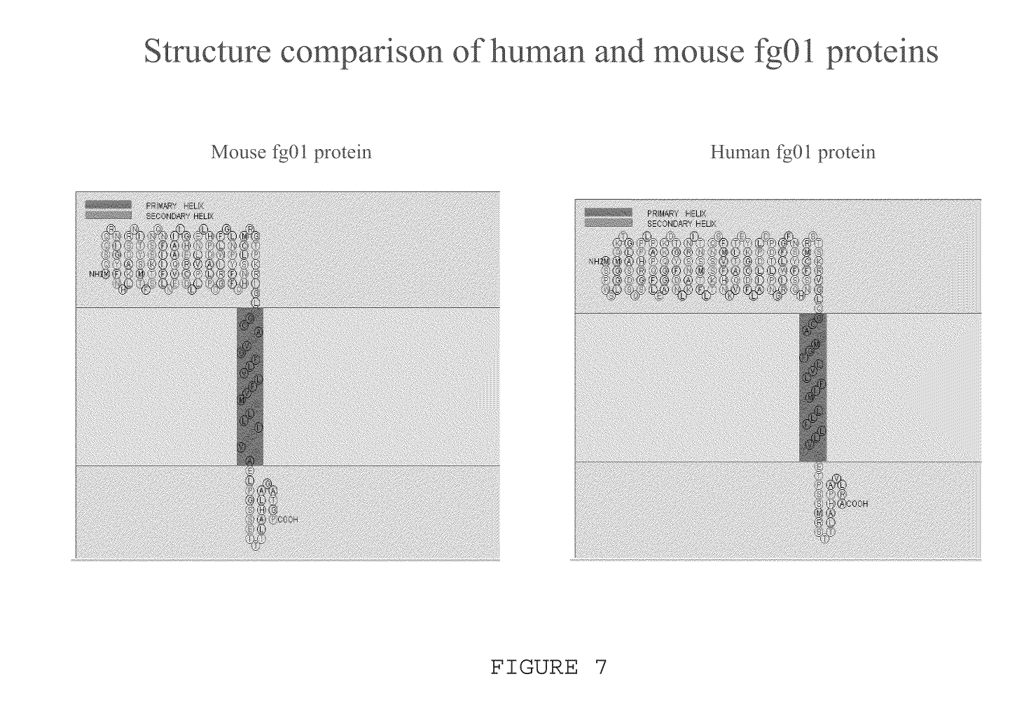

FIG. 8: Truncated Human fg01 EST contig sequence. The sequence was generated by overlapping the human EST sequences. The open reading frame is in blue. The first methionine is underlined in italic and the stop codon is in red.

FIG. 9: Truncated Human fg01 protein sequence. The human fg01 protein sequence was deduced from the EST contig sequence. The first methionine of the protein is in red.

FIG. 10: Alignment analysis of human and mouse fg01 proteins. The truncated fg01 proteins from human and the non-truncated mouse were compared by alignment analysis. The transmembrane domain of the mouse fg01 was boxed.

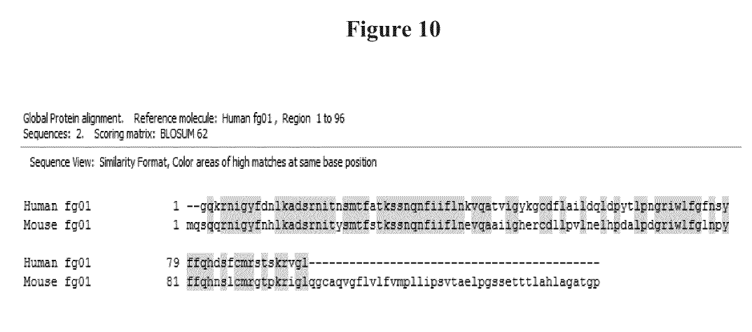

FIG. 11: Alignment analysis of truncated human and chimpanzee fg01 proteins. The truncated fg01 proteins from human and fg01 chimpanzee (Pan troglodytes) protein were compared by alignment analysis. The first methionine of both proteins is boxed.

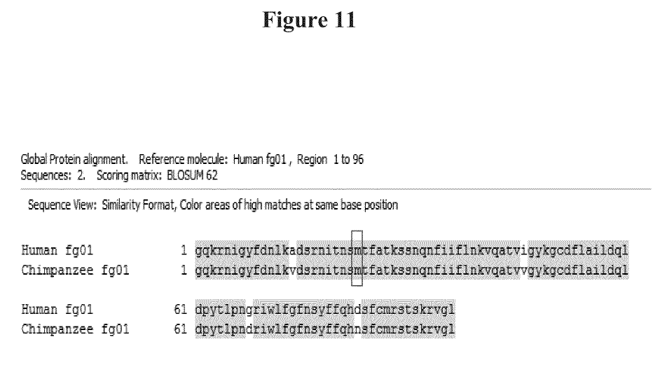

### DESCRIPTION OF THE INVENTION

To identify the human fg01 homolog, the human genome browser search indicated that a genomic DNA domain in human chromosome 8 shares relatively high homology with the mouse fg01 coding sequence. Although there is no human mRNA and EST sequence information available in that locus in GeneBank, the 5′ and 3′ cDNA sequences were amplified by PCR from a human fetal brain cDNA library constructed in a cloning vector using the human chromosome 8-specific primers along with the primers designed from cDNA cloning vector. A 1569-bp full-length cDNA sequence was reconstituted from the PCR products. The cDNA sequence was also confirmed by a separate RT-PCR from a total RNA of human fetal brain.

For clarity, the non-truncated or human fg01 gene and encoded protein are discussed first, herein below. Thereafter, this application addresses the truncated human fg01 protein on chromosome 5, and its corresponding uses.

Human fg01 (Transmembrane Form)

The cDNA sequence perfectly matches human chromosome 8 and contains 4 exons spanning a 6.7 kb genomic contiq. The first exon is located within the CpG island region. An open reading frame encoding a 173 amino acid protein is located within exon 2 and 3. Similar to the mouse fg01 protein, human fg01 homolog is a type 1b transmembrane protein. Human and mouse fg01 proteins share a degree of homology somewhat less than 70%.

Three potential glycosylation sites are identified in the extracellular domain of the human fg01 protein by a computer prediction program. In addition, the computer prediction program also identified several potential phosphorylation sites in both intracellular and extracellular domains of the protein. Similar transmembrane proteins are activated by phosphorylation intracellularly, causing a change in conformation and sometimes activity.

The database search revealed that three SNPs (Single-Nucleotide Polymorphisms) are involved in the coding region leading to 2 amino acids changed. These amino acid changes may be related to the AD pathogenesis, and in particular, presentation of the AD phenotype.

Enhancing expression of the fg01 gene is indicated to be effective in suppressing development of the AD phenotype. In particular, enhanced expression (over expression) of the fg01 gene delays and reduces the formation of plaques commonly associated with AD. The presence of fg01 protein may well suppress the abnormal cleavage of APP leading to accumulation of Aβ and phosphorylation of tau. This offers several different embodiments for intervention to either delay or prevent AD onset, or treat AD to prevent the symptoms from progressing. Methods of enhancing expression of a gene through targeted gene therapy are well known.

In a first alternative, human fg01 could function identically to murine fg01 as described in U.S. patent application Ser. No. 12/399,850. This could arise if fg01 interacts with the cell membrane as a peripheral membrane protein or if it interacts with the cell membrane indirectly via other proteins (e.g., cis-interactions with membrane spanning proteins or via post-translational modifications (e.g., myristoylation) that facilitate membrane interactions. In this scenario, the strategies generally employed to enhance gene expression, through gene therapy, may be used. Thus, the cell genome may be transformed to include multiple copies of the gene, either by transfection with a plasmid incorporating the human fg01 gene operatively linked to a regulatory sequence which enables its expression (e.g., a promoter) or inserted downstream of an active promoter. The cell may be modified to include an amplifiable gene such as DHFR, and exposed to stress such as a toxin like methotrexate to induce amplification of the amplifiable gene and those in its vicinity, which would include the fg01 of the native genome, the DHFR gene having been placed in proximity to the fg01 gene.

Alternatively, gene expression may be upregulated by insertion of promoter and/or enhancer elements up-or-downstream of the genomic transcript, enhancing expression of the gene. These and other methods of enhancing expression through alteration of the human genome, either by insertion of copies of the gene, or alteration of the genome to enhance expression of the gene, are set forth in U.S. Pat. No. 5,272,071, which is incorporated herein-by-reference.

Gene therapy to enhance the expression levels of fg01 protein may be effected in vivo, by introducing transfection plasmids into the host organism cells. Alternatively, they may be effected ex vivo, wherein host cells are transformed in vitro and then introduced back into the host. And of course, they may be effected in vitro.

In addition, if human fg01 protein interacts with the outer cell membrane as a peripheral membrane or soluble (not directly attached to the membrane but via interactions with other proteins, then ectopic delivery of fg01 protein might be sufficient to mediate the same types of effects observed via overexpression of membrane-associated (murine) fg01. In this case, treatment of patients with wild type or preferably recombinant human fg01 could have utility for treating Alzheimer's disease or other indications associated with deposition of Aβ. Likewise, derivatives of human fg01 (e.g., fusion proteins) could serve the same purpose.

Human fg01 may mediate its effects via trans interactions to other membrane associated proteins (much like a soluble growth factor stimulates its receptor) and that this activity might be mimicked using other means to stimulate its ligands. In this case, small molecule (chemical entities, aptamers) or biologics (e.g., antibodies, avimers) that stimulate the same receptor or signaling system could have utility for the treatment of Alzheimer's Disease. In particular, the generation of suitable antibodies using either conventional host immunogenic generation as taught by Kohlerr-Milstein, followed by humanization of the CDRs, or phage display, to provide human antibodies, may be utilized. Antibodies effective therapeutically with other transmembrane proteins, such as the antibodies in Herceptin® and Avastin® are known to those of skill in the art.

Truncated Human fg01 (Chromosome 5)

As with the transmembrane gene found on Chromosome 8, the truncated human fg01 gene found on chromosome 5 offers opportunities not only for diagnostic assays, in identical form to those discussed above for human fg01, but therapeutic intervention with respect to AD, both in those evincing AD phenotypes, and those identified, on the basis of diagnostic assays, as likely AD candidates. This molecule shares sequence homology with murine FG01 but appears to lack a transmembrane domain. Consequently, there are two potential outcomes that could arise from this difference.

In the First Scenario, truncated human FG01 could function identically to murine FG01. This could arise if truncated human FG01 interacts with the cell membrane as a peripheral membrane protein or if it interacts with the cell membrane indirectly via other proteins (e.g., cis-interactions with membrane spanning proteins or via post-translational modifications (e.g., myristoylation) that facilitate membrane interactions. In this scenario, the same types of strategies described above for human FG01 could have applicability to truncated human FG01.

In addition, if truncated human FG01 interacts with the outer cell membrane as a peripheral membrane or soluble (not directly attached to the membrane but via interactions with other proteins, then ectopic delivery of truncated human FG01 might be sufficient to mediate the same types of effects observed via overexpression of membrane-associated human FG01. In this case, treatment of patients with recombinant FG01 could have utility for treating Alzheimer's disease or other indications associated with deposition of A-beta. Likewise, derivatives of truncated human FG01 (e.g., fusion proteins) could serve the same purpose.

The truncated human FG01 might mediate its effects via trans interactions to other membrane associated proteins (much like a soluble growth factor stimulates its receptor) and that this activity might be mimicked using other means to stimulate its ligands. In this case, small molecule (chemical entities, aptamers) or biologics (e.g., antibodies, avimers) that stimulate the same receptor or signaling system could have utility for the treatment of Alzheimer's Disease.

In a Second Scenario, the soluble nature of truncated human FG01 could function as an antagonist of a membrane-associated form of a system analogous to murine FG01. Stated in another way, a membrane associated complex that promotes downregulation of A-beta would not involve truncated human FG01 but instead would be transmitted by a molecule with a “beneficial” function that is analogous to that of human FG01.

In this case, soluble truncated human FG01 could function as an antagonist of the beneficial signaling system. Consequently, therapeutic applications would seek to block truncated FG01. Such therapies could include direct inhibitors of truncated human FG01 expression (e.g., via siRNA or antisense RNA), inducers of truncated human FG01 degradation or elimination (e.g., directed activity of proteases or inhibition of truncated human FG01 secretion) or by therapeutics that prevent soluble truncated human FG01 from functioning as an antagonist of the “beneficial” signaling system that ultimately serves to inhibit A-beta production. Such therapeutics could include antibody-based inhibitors, small molecules or other technologies (e.g., aptamers, avimers) that could interfere with truncated human FG01 function.

Fundamentally, the identification of the human fg01 gene and the fg01 protein encoded, as well as truncated human fg01 and its shorter encoded protein, opens the door for enhanced treatment of Alzheimer's Disease, and enhanced diagnostics. Current diagnostics are largely based on partially subjective testing—degree of loss of cognitive function, higher order reasoning and the like. Although the presence of a large or pronounced amount of plaques and tangles, i.e., the presentation of the AD phenotype, may be indicative of progressive AD, identification of the norm for any particular subject, or the baseline for a population, remains elusive.

By screening for the expression of human and truncated human fg01, as well as the presence of the human and truncated human protein fg01, one can identify those patient's at a potentially higher risk of developing AD. Patients presenting with developing symptoms may be screened for level of human and truncated fg01 in the brain and body, which may allow rapid identification of those whose progression toward profound AD whom might be better or more immediately treated with therapeutics that delay the onset of AD symptoms, like Aricept® (donepezil hydrochloride). The same screening may allow the identification of preferred routes of treatment, either through gene therapy, or through administration of human or truncated human fg01 protein, or a small molecule or biologic which enhances the action of human or truncated human fg01, depending on the frequency of fg01 transcripts, mRNA concentrations, circulating protein levels and the like.

The invention disclosed herein resides in the identification of novel gene sequences and protein sequences. These genes and proteins occur naturally, and thus the invention herein resides in their identification as isolated and detectable forms. By isolated, the inventors herein intend that the indicated nucleic acid sequence, or amino acid sequence, has been separated from the chromosome on which it is found (Chromosome 8 for human fg01, Chromosome 5 for truncated human fg01) or from the cytoplasm and cell and cell debris found in the extracellular matrix, such that the nucleic acid or protein can be identified and manipulated. Purification to a therapeutic level is contemplated, but not a requirement for this invention, or its use.

This invention has been described in terms of the nucleic acid sequence for the identified gene, and the amino acid sequence of the corresponding protein. Those of skill in the art are well aware of alterations to the nucleotide sequence that may be introduced without affecting the protein expressed, including truncation methods, and base alterations that lead to enhanced expression through selection of preferred codons. By the same token, amino acid substitutions that do not disturb the hydropathic index of the human fg01 protein, or truncate those portions of the molecule not involved in binding or structure determination, are familiar to practitioners in this art. These alterations are art of the invention as realized, and within the scope of the claims presented below, unless expressly excluded by the language of the claims.

What is claimed is:

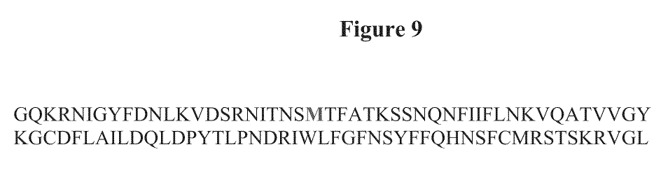

1. An isolated nucleic acid that encodes a protein having the 74 amino acid residue sequence of FIG. 9, or a sequence which exhibits the same conformational structure and biological properties as said sequence of FIG. 9.

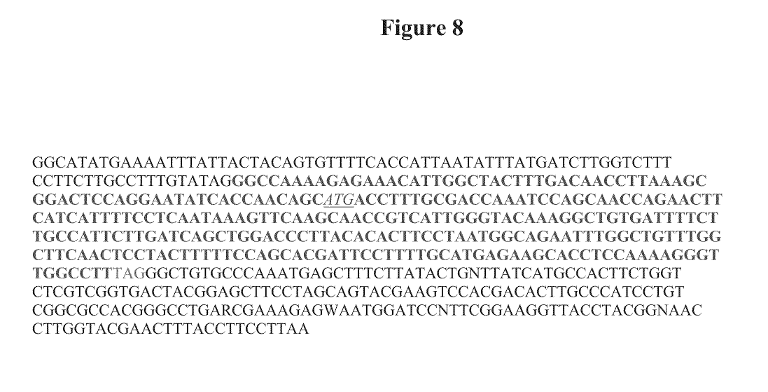

2. The nucleic acid of claim 1, wherein said nucleic acid has the sequence of FIG. 8, or a sequence which varies from said sequence of FIG. 8 by nucleotide bases which do not alter a protein encoded thereby.

3. An isolated nucleic acid which comprises the sequence of FIG. 8.

4. The nucleic acid of claim 3, wherein said nucleic acid has the sequence of FIG. 8.

5. An isolated protein having the amino acid sequence encoded by the nucleic acid of FIG. 8.

6. The protein of claim 5, wherein said protein comprises the 74 amino acid residue sequence of FIG. 8.

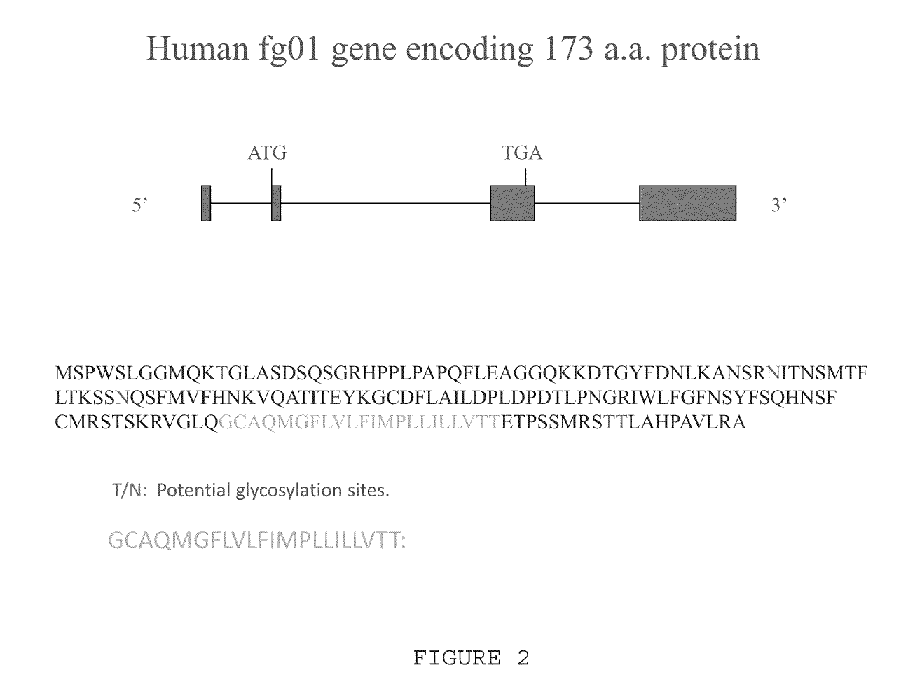

7. The protein of claim 6, wherein said protein has the 74 amino acid residue sequence of FIG. 2.

8. A method of detecting the predisposition of an individual to develop symptoms of Alzheimer's Disease, comprising screening said individual to determine whether the truncated human protein fg01 is expressed at a level below a reference level that is based on the expression of truncated human fg01 in the population from which said individual is drawn, wherein a depressed expression of truncated human fg01 is indicative of a predisposition to development of symptoms of Alzheimer's Disease.

9. A method of suppressing the formation of plaques of amyloid-β in human brain cells, comprising increasing the level of truncated human fg01 protein in said human brain cells.

10. The method of claim 9, wherein said method is effected in vivo.

11. The method of claim 9, wherein said method is effected ex vivo.

12. The method of claim 9, wherein said method is effected in vitro.

13. The method of claim 9, wherein said step of increasing the level of truncated human fg01 protein is achieved by gene therapy resulting in the enhanced expression of truncated human fg01 by cells in a living human.
